(19) United States
(12) Patent Application Publication
(10) Pub. No.: US 2015/0190198 A1
(43) Pub. Date: Jul. 9, 2015

(54) OSTOMY DISPOSAL DEVICE

(71) Applicant: Simon Debel, Bronshoj (DK)

(72) Inventor: Simon Debel, Bronshoj (DK)

(21) Appl. No.: 14/417,805

(22) PCT Filed: Aug. 1, 2013
(86) PCT No.: PCT/EP2013/066153

§ 371 Date: Jan. 28, 2015

(30) Foreign Application Priority Data
Aug. 1, 2012 (DK) PA 2012 00483
Sep. 7, 2012 (DK) PA 2012 00552

(51) Int. Cl. A61B 19/02; A61F 5/448; A61F 5/445

(52) U.S. Cl. CPC A61B 19/029; A61F 5/445; A61F 5/448; A61B 2019/0201; A61B 2019/0267

(57) ABSTRACT

An ostomy disposal device for the disposal of an used ostomy appliance comprising: a first wall and a second wall defining a compartment for holding a used ostomy collecting bag, an proximal end having an opening for providing fluid communication to the compartment, a distal end that is closed, a first closing means arranged distal from the opening of the proximal end for sealing fluid communication between the compartment and the opening, where the proximal end comprises an attachment means for attaching the bag to a user and where the attachment means is adapted to provide a liquid and air tight seal between the skin surface of the user and the opening.

### FIELD OF THE INVENTION

An ostomy disposal device for the disposal of a used ostomy appliance comprising: at least one layer of sheet material defining a compartment for holding a used ostomy collecting bag, a proximal end, a distal end that is closed, an opening for providing fluid communication to the compartment, a first closing means arranged distal from the opening of the proximal end for sealing the fluid communication between the compartment and the opening, where the proximal end comprises an attachment means for attaching the bag to a user.

### BACKGROUND

In relations to surgery for diseases in the gastro-intestinal tract, many patients may be left with an abdominal stoma such as a colostomy, ileostomy or a urostomy, where the surgeons create an artificial opening into the intestines or the urinary tract. The artificial opening may temporarily or permanently replace a natural opening into the intestines or the urinary tract and is used for the removal of bodily waste, such as feces or urine.

Users having an abdominal stoma are often provided with a collecting bag, which collects the bodily waste, as users having an artificial stoma usually do not have voluntary control of the release of their bodily waste. Thus, the collecting bag is provided surrounding the stoma in order to ensure that the bodily waste is collected.

In case of a colostomy or a ileostomy, the collecting bag is used to accumulate excrements that may be soft, runny or in liquid form, depending on where the stoma is introduced to the intestines or the bowels. Furthermore, the excrements may have an unpleasant odour, when removed from the body, as the excrements may be removed from the body at an early stage, where the body has not finished its treatment of the excrements, when comparing to a healthy individual. This means that there may be acids, enzymes, bacteria, or other compounds present in the excrements that increase the undesired odour, compared to normal and healthy excrements.

The unpleasant odour of excrements from a stoma is an issue that has been historically addressed by the makers of ostomy collecting bags, where the material of the ostomy bag is often impermeable to the unpleasant odour, and the bag's coupling means are made in such a way that it is difficult for odour to escape via the adhesive coupling that adheres the ostomy collecting bag to the body of the user.

An ostomy collecting bag is connected to the body of the patient, where the ostomy bag may provide a hermetic and a liquid proof seal around the user's stoma, ensuring that the excrements and the unpleasant odour do not escape the ostomy bag during use.

However, when the user removes and/or replaces a used ostomy collecting bag, the user will break the hermetic or liquid proof seal, allowing access to the stoma to the ambient surrounding air. When the user has removed the ostomy collecting bag from the body, there may be residual excrements on the skin surface surrounding the stoma and/or on the parts of the stoma that extrude from the abdominal skin surface. Thus, the user must clean the stoma and the surrounding areas in order to prevent or to reduce that the residual excrements irritate and/or damage the surrounding skin. Such cleansing is quite important for the user, as the skin surrounding the stoma is quite sensitive to the effluents exiting the stoma and is a major cause of complication for the users.

Currently, the users usually enter a bathroom facility and will take a wash kit with them. The wash kit will usually contain wet wipes and dry wipes for cleaning the stoma and for drying it afterwards. The user will remove or displace the articles of clothing that cover the stoma and the ostomy collecting bag, and subsequently remove the collecting bag from the skin surface, and thus exposing the stoma. Upon removal of the collecting bag, the user will start by cleaning the stoma and the surrounding areas with wet cloths that are intended to facilitate the removal of residual effluents and excrements from the stoma and surrounding areas, where the user follows up by patting the area dry afterwards before replacing the used collecting bag with a clean collecting bag.

The process of cleaning the stoma may be time consuming and difficult, especially when the user has to perform the cleaning in a public restroom/toilet, where the user does not have all the necessary tools or material at hand for cleaning the stoma. Thus, the user will have a kit of articles that are useful for cleaning the stoma which may further contain a bag for disposing the used wipes and/or the used collecting bag. Due to the fact that the restroom facilities may be of a kind that do not have a wash basin or a sink or any areas for positioning the disposal bag during the cleaning of the stoma, this may cause an inconvenience for the user when cleaning the bag, as the user may have to position the bag at a distance from the stoma, such as placing it on the lid of a toilet or alternatively on the floor.

The positioning of the disposal bag may be inconvenient for the user, as the risk of contaminating the users clothing is increased when the user has to manoeuver the cloths from the stoma and towards the bag for disposal, creating a risk that the used cloths or the exposed stoma may contaminate the user's clothes or surrounding area.

Thus, there is a need to facilitate and improve the change of a used ostomy collecting device and/or bag, where the risk of contamination is reduced when the user is not in an optimal surroundings or environment for cleaning the stoma, such as in a public restroom.

### GENERAL DESCRIPTION

In accordance with the invention, there is provided an ostomy disposal device for the disposal of a used ostomy appliance comprising: at least one layer of sheet material defining a compartment for holding a used ostomy collecting bag, a proximal end, a distal end that is closed, an opening for providing fluid communication to the compartment, a first closing means arranged distal from the opening of the proximal end for sealing the fluid communication between the compartment and the opening, where the proximal end comprises an attachment means for attaching the bag to a user and where the attachment means is adapted to provide a liquid and air tight seal between the skin surface of the user and the opening.

The ostomy disposal device may be attached to the user, in order to allow the disposal of a used ostomy appliance directly into the compartment of the ostomy disposal device, via the opening. Such a device facilitates an easier removal of a used ostomy appliance, as the opening of the device may be placed in the vicinity of the ostomy base plate that is positioned on the user, advantageously positioned beneath the adhesive base plate, so that the distance between the opening and the stoma is as small as possible. This reduces the risk that exudates from the stoma or the used appliance will contaminate the user or the surroundings when removing the ostomy device.

The user will disconnect the ostomy appliance from the body, and introduce the appliance into the compartment of the device, and may further clean the area surrounding the stoma as well as the stoma using wet and/or dry wipes, and place those wipes directly into the compartment of the device. When the user has finished removing the ostomy appliance, and may be ready to attach an unused ostomy appliance, the sealing means may be used to close the compartment of the device, and sealing the contaminated articles placed inside the compartment such that odour, gasses and/or liquids inside the compartment are maintained inside the compartment.

Subsequently, the attachment means of the ostomy disposal device may be disconnected, and the disposal device may be disposed of into the garbage bin or other suitable disposal places.

By utilizing such a device, the user may easily dispose of a ostomy appliance in surroundings where there are inadequate facilities for changing and/or disposing of a ostomy appliance, such as in a public restroom, or in areas where there is no access to bathroom facilities. Should the user be in inadequate facilities during the changing of an ostomy appliance, the user may have to place the used ostomy bag on e.g. a toilet seat, the ground, or other accessible surfaces, while the stoma and its surrounding areas is being cleaned. Such placement movement of the user may mean that the user has to bend from the hip, which increases the risk that the uncleaned stoma may contaminate the user's clothes or other skin surfaces as the bending movement may move the article of clothing or a skin fold onto towards the stoma.

Furthermore, when the ostomy disposal device is attached to the user, the user has both hands free to remove the ostomy appliance and to clean the stoma, which is different from a conventional disposal bag, which has to be held close to the stoma, should the user wish to have a disposal bag close to the stoma when removing the used ostomy device.

Thus, the user can ensure that the used ostomy bag is responsibly disposed of, the distance the contaminated ostomy appliance has to be manoeuvred may be significantly reduced, and that the user may substantially be in the same body position during the change of the ostomy appliance.

In situations where the user may be in a public restroom, or a toilet stall, it may be advantageous that the odour that escapes the used ostomy appliance when disconnected from the user's body is contained inside the ostomy disposal device, so that the odour does not cause the user unwanted embarrassment in a public place. Thus, the attachment means may be adapted to provide a fluid tight seal between the skin surface and the opening, and thus the compartment of the bag. This means that the user can position the ostomy disposal device around the used ostomy appliance, so that the disposal device contains the entire ostomy appliance and possibly also the ostomy base plate (for one piece ostomy appliances). Thus, the ostomy disposal device may hermetically isolate the ostomy appliance from its surroundings, so that when the seal between the ostomy appliance and the skin and/or the base plate the odour of the exudates is contained inside the ostomy disposal device.

The released ostomy appliance may subsequently be manoeuvred into the compartment of the ostomy disposal device and the sealing means may be used to close off the compartment holding the used ostomy appliance, and thereby ensuring that odour producing exudates in the ostomy appliance is sealed inside the compartment. Subsequently, the ostomy disposal device may be released from the user, breaking the liquid and air tight seal between the skin surface of the user and the opening.

This means the user is capable of exchanging the ostomy appliance without exposing the used ostomy appliance to the ambient air, and thereby reducing the risk that unpleasant odour is released into the atmosphere.

Within the meaning of the present invention, an ostomy base plate or a flange may be synonymous with the skin surface of the user, as the ostomy base plate may be a long term base plate that is not intended to be changed with each used ostomy collecting bag. Such a system may be a 2 piece ostomy appliance where the ostomy collecting bag may be changed at a higher frequency than the ostomy base plate. Thus, the attachment means may be attached to the ostomy base plate, in case the ostomy collecting bag may be separated from the ostomy base plate when changed.

In one embodiment of the invention the opening may be an annular opening. The opening of the bag may be formed from one or two of the walls of the bag, where the annular opening may be cut into the walls, creating an annular opening. The annular opening may be in annular form when the opening is extended into its ultimate position, so that the ostomy appliance, and optionally the base plate may be inserted into the compartment via the opening in any orientatation. I.e. that it does not matter at which angle the ostomy appliance is turned, provided that the longest axis of the ostomy appliance is not substantially parallel to the radial axis of the opening.

In one embodiment of the invention, the peripheral edge of the opening may be provided with a stiffening ring, which is capable of preventing the opening from collapsing during use. The stiffening ring may be constructed of a resilient memory material, which may be folded or collapsed during storage, but will return to its memory position holding the opening rigid and/or accessible when the disposal bag is in use.

In one embodiment of the invention, the compartment is defined by a first wall and a second wall. The first and the second walls may be joined at a peripheral edge of the first wall and the second wall, in order to collectively define a compartment. The first wall and the second wall may be a front and a back wall, where the back wall faces the user and the front wall faces away from the user when the ostomy disposal device is in use.

In one embodiment of the invention, the proximal end is provided with the opening. By providing the opening close to the proximal end it is possible to maximize the volume of the ostomy disposal device, ensuring that the opening is positioned at a distance from the distal end of the bag, and thereby ensuring that the size of the compartment, between the opening and the distal end is as large as possible, based on the size of the bag. The closing means may therefore be positioned between the distal end and the opening, in order to ensure that the used ostomy collecting device may be accommodated inside the compartment subsequent to the sealing of the closing means, and thereby ensuring that fluid communication is prevented from the compartment and towards the opening.

In one embodiment of the invention the attachment means may be an adhesive layer for attaching the ostomy appliance to the skin surface of the user. When the user is to remove a used ostomy appliance, it is often necessary for the user to remove the clothing article that covers the stoma, often by removing the clothing article completely or alternatively by pushing the clothing article away from the stoma. Thus the skin surface of the user is often exposed, and may therefore be utilized as a surface to which the attachment means may be adhered to. This also means that the user may adhere the ostomy disposal device close to the stoma, e.g. below the base plate, which means that the ostomy appliance may e.g. be partly positioned inside the compartment of the ostomy disposal device, when the appliance is disconnected from the body. This means that the risk of contamination is reduced should the user lose grip of the ostomy appliance, as the bag would simply drop into the compartment of the ostomy disposal device and would not drop to the ground.

The adhesive layer may be in the form of an adhesive strip that is positioned in the vicinity of the periphery of the opening, so that there is a short distance from the edge of the adhesive strip to the periphery of the opening. This means that when the ostomy disposal device is attached to the body of the user, the periphery of the opening is positioned close to the skin surface of the user, in order to minimize the risk that the ostomy disposal device may be contaminated in an area that is between the opening and the adhesive strip. The adhesive strip may in one embodiment be positioned so that the adhesive surface abuts the periphery of the opening of the bag.

In one embodiment of the invention the opening may be adapted to surround a stoma and/or an ostomy base plate worn by the user. I.e. the size of the opening may be such that the opening is large enough to surround the stoma or the base plate, so that the entire base plate may easily be fitted through the opening without having to fold or bend the base plate in order to place the ostomy appliance into the compartment of the ostomy disposal device. The size of the ostomy base plate (flange) may vary from one provider to the other, but the opening may advantageously be of a size between 50 and 300 mm, more particularly between 60 and 250 mm, more particularly between 70 and 230 mm, more particularly between 80 and 210 mm, more particularly between 90 and 190 mm, more particularly between 100 and 170 mm or more particularly approximately 150 mm.

In one embodiment, the diameter of the opening may be smaller than the inner diameter of the compartment. In another embodiment the diameter of the opening may be equal to the inner diameter of the compartment. In another embodiment the diameter of the opening may be larger than the inner diameter of the compartment.

In one embodiment of the invention the attachment means may be arranged at a peripheral edge of the opening. By placing the attachment means at the peripheral edge of the opening, there is no wall material between the attachment means and the opening, reducing the risk of contaminating the ostomy disposal device in other areas than where it is intended, i.e. the opening. This means that when the ostomy disposal device is attached to the body of the user, the periphery of the opening is positioned close to the skin surface of the user, in order to eliminate the risk that the ostomy disposal device may be contaminated in an area that is between the opening and the attachment means.

The attachment means may be e.g. a mechanical attachment means and be in the form of a hook and/or loop attachment, that is capable of being attached using the hook attachment part to a piece of clothing, e.g. to the waist of trousers or similar. The attachment means may further be a loop into which a belt may be introduced, a hooking mechanism that allows the ostomy disposal device to be attached to a protruding part of a clothing (belt loop), the waistband of trousers or similar. The attachment means may further be an adhesive layer, providing an adhesive surface that allows the attachment means to be adhered to a surface area available to the user, such as the skin surface, the outer surface of the ostomy base plate, and any surface of a clothing material. Alternatively the attachment means may be of the kind that allows the ostomy disposal device to be attached to an external surface area in the user's surroundings, such as a wall of a restroom cubicle, to a sink, a toilet or similar allowing the user to position the ostomy disposal device in a practical accessible position, should the user not be inclined to attach the ostomy disposal device to his/her body. Such attachment means may e.g. be an adhesive layer that has enough tack to adhere to a surface and to support the weight of the disposal device and its contents, but low enough tack to be easily removed from the surface it is adhere to without having to assert excessive force. Such considerations should also be applicable to an adhesive layer that is attached to the user, in order to minimize the risk that the user has to experience pain when removing the ostomy disposal device from the user, e.g. from a skin surface.

In one embodiment of the invention, the first closing means comprises a second adhesive layer arranged at an inner peripheral edge of the opening, allowing the inner surface of the sheet material to be adhered to the inner peripheral edge and thereby sealing fluid communication to the compartment from the outside. This means that the second adhesive layer may be exposed, and the inner surface of the opposing sheet material (the sheet material that faces away from the user when the bag is attached to the user) of the ostomy disposal device may be applied to the surface of the adhesive ensuring that fluid communication is closed from the compartment holding the used ostomy device and the opening.

In one embodiment of the invention the attachment means may be in the form of an annular adhesive layer, extending along an entire peripheral edge of the opening. The annular adhesive layer may be adhered to the skin surface of the user, surrounding the base plate. Thus, the annular adhesive layer, and the opening encloses the stoma and the used ostomy appliance, where the annular adhesive layer may provide an air and liquid tight seal between the skin surface of the user and the ostomy disposal device. The annular adhesive layer may be welded, adhered or otherwise attached to the ostomy disposal device, ensuring that the connection between the adhesive layer and the device is air and liquid proof.

Thus, the user can introduce the free end of the ostomy appliance into the opening of the ostomy disposal device, inserting the ostomy collecting bag into the compartment. Subsequently, the user may align the annular adhesive layer around the ostomy base plate, and applying the annular adhesive layer to the skin surface of the user along the entire periphery of the opening, where the annular adhesive layer creates a seal between the ostomy disposal device and the skin surface of the user. Upon attachment, the user may commence on removing the used ostomy collecting bag from the body, where the removal may be performed through the walls of the ostomy disposal device. The walls of the ostomy disposal device may be transparent, in order to allow the user to view the content of the disposal device during removal. When the used ostomy collecting bag has been disconnected from the body, it may be moved inside the compartment towards the closed end of the ostomy disposal device, and subsequently the closing means may be applied in order to enclose the used and contaminated bag inside the compartment. Following that, the user may release the annular adhesive layer from the skin surface and remove the ostomy disposal device from the body. In this way, the used ostomy collecting bag has been removed from the body of the user, without having the used collecting bag exposed to the ambient air, and therefore reducing any awkward or uncomfortable odour inconvenience for the user.

Alternatively, the annular adhesive layer may be adhered to the outer surface area of the base plate surrounding the stoma, with the same effects as above.

In one embodiment of the invention, the compartment of the ostomy disposal appliance may further comprise one or more items selected from the group of, at least one single use glove, at least one wet tissue, at least one dry tissue, at least one disposable cleaning means. This means that the ostomy disposal device may be provided with accessories that are useful for the removal of a used ostomy appliance and/or for the cleaning of an exposed stoma. By arranging the items inside the compartment, the items are always accessible for the user when using the ostomy disposal device to dispose a used ostomy appliance. The single use glove may be used to prevent the user from contaminating his hands with remaining exudates and/or to prevent bacteria from transmitting from the hand to the stoma, and vice versa. The tissues may be provided inside the compartment, so that when the ostomy disposal device is attached to the user, the user has easy access to the items. Furthermore, by providing the items inside the compartment, the user may clean the stoma while the ostomy disposal device seals off the used ostomy bag during removal, so that the stoma may be cleaned while the ostomy disposal device seals off the odour inside the compartment. Thus, the removal of a used ostomy appliance and the cleaning of the stoma may be performed without having an odour release from the exudates of the stoma that are present in the bag or, on and/or around the stoma.

The above items may be placed in one or more small pockets inside the compartment, where the pockets are open, allowing easy removal from the pockets when the items are needed for removal or cleaning.

In one embodiment of the invention, the ostomy disposal device may further comprise a cover bag arranged to enclose at least the distal end of the ostomy disposal device for encompassing the ostomy disposal device in a compacted state. This means that the distal end of the ostomy disposal device may further be provided with a cover bag, where the inner surface of the cover bag may be attached to the outer surface of the distal end, and where the cover bag is formed so that the ostomy disposal device may be folded into a compacted state and placed inside the cover bag. This means that the cover bag may be used as an encapsuling for an unused ostomy disposal device, i.e. prior to use, and where the device may be unfolded from the bag when the user is preparing to use it. Further, the cover bag may be used to enclose a used ostomy disposal device, so that when the used ostomy disposal device contains a used ostomy appliance, the ostomy disposal device may be folded into a compacted state allowing the cover bag to enclose the used device.

In one embodiment, the cover bag may comprise a second closing means, arranged in the vicinity of an opening of the cover bag. Thus, when the used ostomy disposal device has been folded into the cover bag, the cover bag may be closed using the second closing means, in order to ensure that any residue of stoma exudates that may have contaminated the ostomy disposal device may be isolated inside the cover bag. This may increase the sanitary requirements for the user, allowing the user to e.g. carry the compacted device prior to disposal, should the disposal facilities be lacking when the user has changed the used ostomy appliance.

The first and/or the second closing means may comprise a draw band or strap, which may be an elastic or resilient draw band or strap. Furthermore, the said closing means may be in the form of an adhesive that is capable of sealing off the compartment of the ostomy disposal device or is capable of closing off the open end of the cover bag.

In accordance with the invention, there is also provided a method to remove a used ostomy appliance comprising the steps of: providing an ostomy disposal device defining a compartment for receiving an used ostomy appliance, attaching the ostomy disposal device to the user, releasing the used ostomy appliance from the user, introducing the used ostomy appliance into the compartment of the ostomy disposal device, sealing the used ostomy appliance inside the compartment and/or releasing the ostomy disposal device from the user.

In accordance with the invention there is also provided an ostomy appliance comprising an ostomy disposal device as claimed. This means that the ostomy disposal device may be permanently or temporarily attached to the ostomy appliance, in order to ensure that when the user needs a disposal device, the disposal device may be part of the ostomy appliance already attached to the user's body.

In one embodiment of the present invention the ostomy disposal device may be provided with an air filter to allow air to pass from inside the compartment and into the ambient air. The filter may be of the kind that is capable of reducing the unpleasant odours from the excrements or exudates the compartment of the air inside the compartment, when the air is passed through the filter. Such filters may be active charcoal filters that are specifically designed to improve air quality from inside ostomy appliances, such as Coloplast Filtrodor® Ostomy Pouch Filter, or other suitable filters.

In one embodiment of the invention, the first closing means may be arranged at a distance below the opening of the ostomy disposal device and above the distal end equal to at least ¼ or ⅓ of the total length of the first cover bag. It is also preferred that the first closing means may be arranged at a distance above the distal end equal to at least ⅕, ¼ or ⅓ of the total length of the first cover bag. The total length of the ostomy disposal device may be seen as the distance from the proximal end to the distal end of the ostomy disposal device.

In one embodiment of the invention, the ostomy disposal device may comprise an inlet or valve that may be provided at a first or a second wall of the ostomy disposal device for providing access to the inner volume of the compartment. The inlet may e.g. be used for introducing cleaning tissues, creams, or other useful items through the inlet and into the compartment when needed.

### BRIEF DESCRIPTION OF DRAWINGS

The invention is explained in detail below with reference to the drawings, in which

FIG. 1 is an ostomy disposal device in accordance with the invention, shown in perspective,

FIG. 2 is the same shown from the front, in the vicinity of a stoma.

FIG. 3 is the same shown when attached to a user,

FIG. 4 is the same shown when the compartment is sealed.

FIG. 5 is the same shown where the ostomy disposal device is compacted into an outer bag,

FIG. 6 is an embodiment of an ostomy disposal device shown from the front,

FIG. 7 is an embodiment of an ostomy disposal device in accordance with the invention, seen in perspective,

FIG. 8 is the same, shown from the front and attached to a user, and

FIG. 9 is the same, shown in a side sectional view.

### DETAILED DESCRIPTION OF DRAWINGS

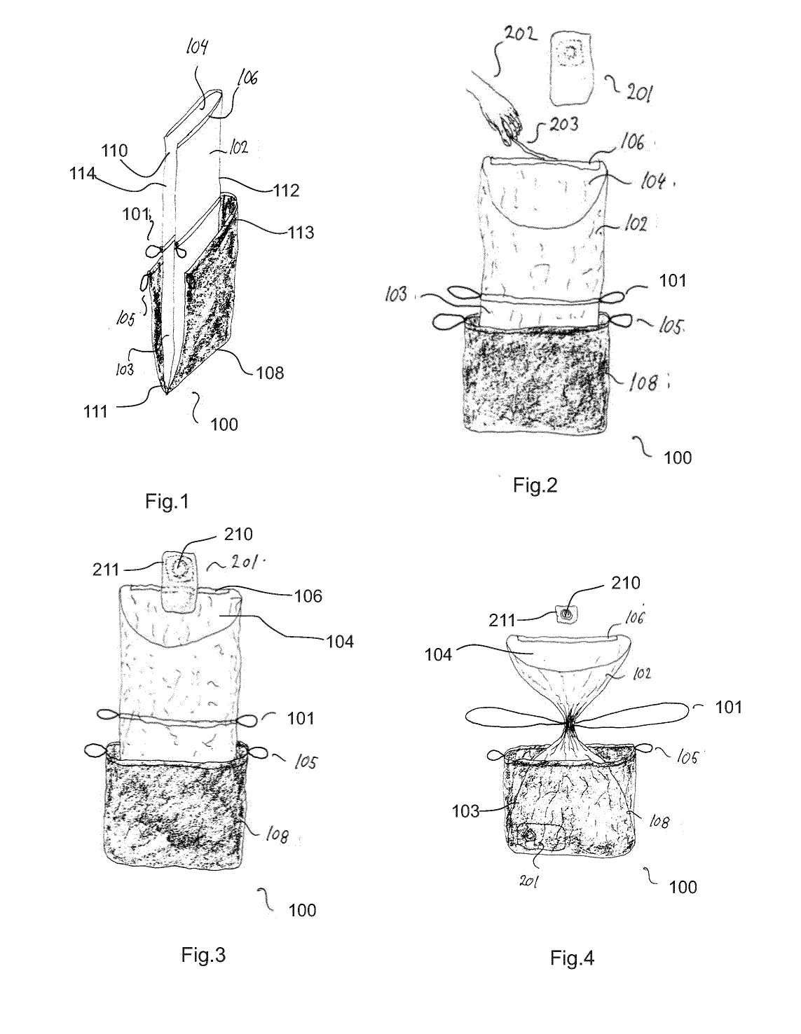

FIG. 1 shows a perspective view of a ostomy disposal device 100 in accordance with the invention, where the ostomy disposal device is folded along its longitudinal axis 112 extending from the proximal end 110 of the device 100 to the distal end 111 of the device 100. The ostomy disposal device comprises a first wall 102 and a second wall 114 that define an inner compartment 103, for storage of a used ostomy appliance (not shown). The ostomy disposal device 100 is provided with a closing means 101, in the form of one or more drawstrings, that allow the compartment 103 of the ostomy disposal device 100 to be sealed off in an air and liquid tight manner, to prevent and/or reduce the release of exudates or odours from the closed off compartment 103. It may be preferred that the distance from the opening 104 to the first closing means 101 is at least ¼ or ⅓ of the total length between the proximal end 110 ant the distal end 111, and where it may be preferred that the distance from the distal end 111 to the first closing means 101 is at least ⅕, such as at least ¼, or such as at least ⅓ of the total length between the proximal end 110 ant the distal end 111.

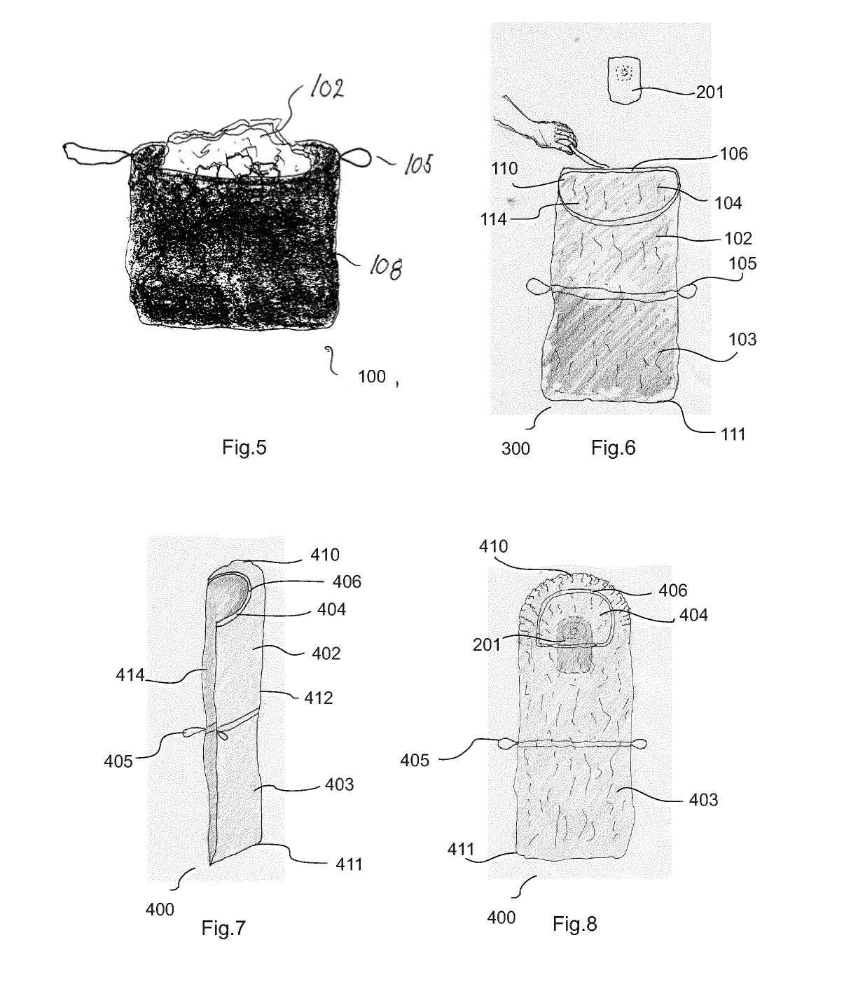

The ostomy disposal device 100 in accordance with this embodiment is provided with a cover bag 108, which encloses the distal end 111 first 102 and second walls 104 of the ostomy disposal device 100. The cover bag 108 may be attached to the distal end 111, in order to ensure that the cover bag 108 does not separate from the distal end 111 prior to, during and/or after use of the ostomy disposal device 100. The proximal end 113 of the cover bag 108 may be provided with a second closing means 105, in the form of a drawstring that is adapted to seal off the inner volume of the cover bag 108 when a used ostomy appliance has been introduced into the compartment 103, and the proximal end 110 has been folded into the inner volume of the cover bag, as shown in FIG. 5.

The proximal end 110 of the ostomy disposal device 100 may be provided with an attachment means 106, in the form of an adhesive layer that extends along the peripheral edge of the opening 104 into the compartment 103, and is shown in more detail in FIG. 2. The user may position the ostomy disposal device 100 in the vicinity of an ostomy appliance 201, and remove a release liner 203 from the adhesive layer 106, and thus preparing the ostomy disposal device 100 for use.

The ostomy disposal device 100 may be positioned in the vicinity of the stoma 210, as seen in FIG. 3, where the opening 104 and the adhesive layer 106 is positioned slightly below the periphery of the ostomy base plate 211 and is attached to the skin surface of the user. The ostomy appliance 201 may be partly introduced into the opening 104 of the ostomy disposal device 100, ensuring that when the appliance 201 is released; gravity will feed the appliance 201 into the compartment 103 of the device 100.

FIG. 4 shows the ostomy disposal device 100 when the ostomy appliance 201 has been removed from the stoma 210 and/or the ostomy base plate of the user. The ostomy appliance 201 has been introduced into the compartment 103, ensuring that any exudates from the used ostomy appliance be kept inside the compartment. The first closing means 101 may be applied, where a closing means seal the compartment 103 from the opening 104, ensuring that the air and the liquids inside the compartment cannot communicate with the opening, and thereby ensuring that the contents of the compartment 103 remain inside the compartment. This closing action of the first closing means 101 may be performed while the device 100 is still attached to the body via the attachment means 106, or may alternatively performed after the device 100 has been released from the body.

Subsequent to closing the first closing means 101, the ostomy disposal device 100 may be released from the body, and the closed compartment and the proximal end 110 may be folded into the inner volume of the cover bag 108 so that the first 102 and second wall 114 are fully introduced into the cover bag, as shown in FIG. 5. The second closing means 105 may subsequently be activated to close off the inner volume of the cover bag 108, providing a small package that may easily be disposed of, without risking that any exudates or contamination that is present on or near the opening or on the first or second wall of the device 100 is closed off inside the cover bag 108. Thus, the user is provided with a hygienic package that may be introduced into disposal containers, without risking a cross contamination of stoma exudates and/or without emitting unpleasant smells in public areas.

FIG. 6 shows an embodiment of the invention, where the ostomy disposal device 300 is provided similar to that shown in FIGS. 1-5, where the cover bag 108 has been removed. The reference numbers of the parts of the device 100 in FIG. 1-5 are the same for ostomy disposal device 300. This device may be used in a similar manner as that shown in FIG. 1-5, where the used ostomy appliance 201 may be introduced into the compartment 108, and the first closing means 105 may be used to seal off the compartment 108 from the opening 104, ensuring that the seal is liquid and/or air tight, and preventing liquid and air contamination from the inner volume of the compartment 108, when subsequent to the use of the device 300.

The devices shown in FIG. 1-6 are particularly advantageous when exchanging a ostomy appliance in an unfamiliar environment, where the user does not have access to optimal facilities to dispose an ostomy appliance and to clean the stoma.

FIG. 7 shows an embodiment of an ostomy disposal device 400, where the opening 404 of the device 400 is an annular opening, having an adhesive layer 406 extending along the entire periphery of the opening 404. The device 400 is seen as folded along a longitudinal axis 412 of the device 400, where the longitudinal axis extends from the proximal end 410 of the device 400 to the distal end 411 of the device 400. The device is provided with a first closing means 405, that functions similarly to the first closing means shown in FIG. 1-6, and is capable of sealing off the compartment 403 of the device 400 in an air and liquid tight manner, ensuring that the contents of the compartment (when filled with items to be disposed of have been introduced into the compartment 403.

The opening 404 of the device 400 may be seen as an annular opening 404 that has an annular peripheral edge, that defines the opening of the device 400 into the compartment 403 allowing fluid communication from the outside of the device 400 and into the inner volume of the device 400, i.e. the compartment 403. The peripheral edge of the opening 404 may be provided with an annular adhesive surface 406 that extends along the entire peripheral edge of the opening 404.

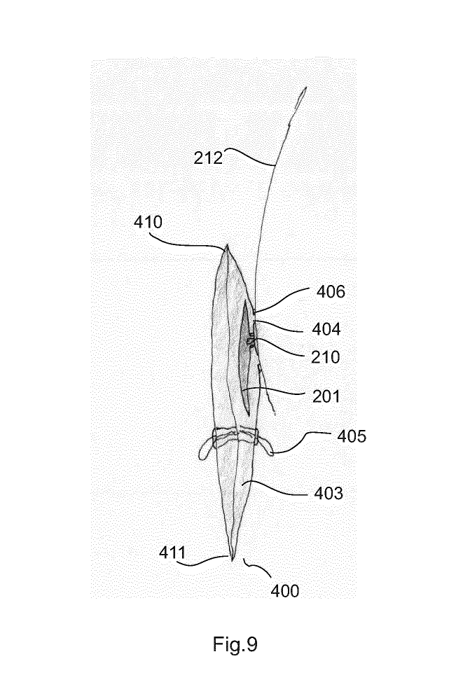

FIGS. 8 and 9 shows a frontal and side sectional view of the device 400, respectively, where the annular opening 404 has been adhered to the skin surface of the user using the annular adhesive layer 406 arranged at the periphery of the opening 404. The opening 404 of the device 400 has been arranged around the ostomy appliance, introducing the ostomy appliance 201 into the inner volume of the device 400, i.e. the compartment 403, or into the inner volume of the device that communicates with the compartment 403. The annular adhesive layer 406 ensures that there is an air and liquid proof seal between the opening 404 and the skin surface of the user, so that the ostomy appliance 201 may be removed/released from the body, without exposing the inner content of the appliance 201 to the ambient air. Thus, it is possible to prevent the odour coming from the used appliance from contaminating the air of a public bathroom or similar areas where the odour may cause the user embarrassment or be uncomfortable to the user.

The ostomy disposal device 400 may therefore be used to isolate the stoma 210 and the ostomy appliance 201 from the ambient air, ensuring that when the ostomy appliance is being released from the skin surface 212 any contaminants are kept inside the inner volume of the device 400. When the ostomy appliance 201 has been removed from the skin surface 212, the ostomy appliance may be introduced into the compartment 403 and the first closing means 405 may be applied, ensuring that the contents of the compartment 403 are maintained inside the compartment 403.

In an alternative embodiment of the ostomy disposal device 400 in accordance with the invention, a cover bag 108 similar to that shown in FIG. 1-5, may be attached to the distal end 411 of the device 400, and function in a similar manner to that disclosed in relation to FIGS. 1-5.

In accordance with the invention, the entire contents or parts of the Danish patent applications PA 2012 00552 and PA 2012 00483 is hereby incorporated by reference into the present application.

1. An ostomy disposal device for the disposal of a used ostomy appliance comprising
at least one layer of sheet material defining a compartment for holding a used ostomy collecting bag,
a proximal end,
a distal end that is closed,
an opening for providing fluid communication to the compartment,
a first closing means arranged distal from the opening of the proximal end for sealing the fluid communication between the compartment and the opening,
where the proximal end comprises an attachment means for attaching the bag to a user,
characterized in that the attachment means are adapted to provide a liquid and air tight seal between the skin surface of the user and the opening

2. An ostomy disposal device according to claim 1 where the opening is an annular opening.

3. An ostomy disposal device according to claim 2 wherein the peripheral edge of the opening is provided with a stiffening ring, which is capable of preventing the opening from collapsing during use.

4. An ostomy disposal device according to claim 1 wherein the compartment is defined by a first wall and a second wall.

5. An ostomy disposal device according to claim 1 wherein the opening is provided at the proximal end of the device.

6. An ostomy disposal device according to claim 1 where the attachment means is an adhesive layer for attaching the ostomy appliance to the skin surface of the user.

7. An ostomy disposal device according to claim 1 where the opening is adapted to surround a stoma and/or a stoma base plate worn by the user.

8. (canceled)

9. An ostomy disposal device according to claim 5 wherein the attachment means are arranged at a peripheral edge of the opening.

10. An ostomy disposal device according to claim 1 wherein the attachment means is in the form of an annular adhesive layer, extending along an entire peripheral edge of the opening.

11. An ostomy disposal device according to claim 1 wherein the compartment of the ostomy disposal appliance may further comprise one or more items selected from the group of, at least one single use glove, at least one wet tissue, at least one dry tissue, at least one disposable cleaning means.

12. An ostomy disposal device according to claim 1 wherein the first closing means comprises a second adhesive layer arranged at an inner peripheral edge of the opening, allowing the inner surface of the sheet material to be adhered to the inner peripheral edge and thereby sealing fluid communication to the compartment from the outside.

13. An ostomy disposal device according to claim 1 wherein the ostomy disposal device may further comprise a cover bag arranged to enclose at least the distal end of the ostomy disposal device for encompassing the ostomy disposal device in a compacted state.

14. An ostomy disposal device according to claim 1 wherein the ostomy disposal device further comprises a cover bag arranged to enclose at least the distal end of the ostomy disposal device for encompassing the ostomy disposal device in a compacted state.

15. A method of removing a used ostomy appliance comprising the steps of:
providing an ostomy disposal device defining a compartment for receiving an used ostomy appliance,
attaching the ostomy disposal device to the user,
releasing the used ostomy appliance from the user,
introducing the used ostomy appliance into the compartment of the ostomy disposal device,
sealing the used ostomy appliance inside the compartment and/or releasing the ostomy disposal device from the user.
